(19) United States
(12) Patent Application Publication
(10) Pub. No.: US 2007/0179493 A1
(43) Pub. Date: Aug. 2, 2007

(54) Magnetic spinal implant device

(76) Inventor: Richard C. Kim, San Diego, CA (US)

Correspondence Address: Richard C. Kim, 13023 Polvera Avenue, San Diego, CA, 92128, US

(21) Appl. No.: 11/652,792

(22) Filed: Jan. 12, 2007

Related U.S. Application Data
(60) Provisional application No. 60/759,094, filed on Jan. 13, 2006.

(51) Int. Cl. A61B 18/04

(52) U.S. Cl. 606/33

(57) ABSTRACT

A magnetic spinal implant device is disclosed. In one embodiment, the device includes: a first piece configured to be implanted into a patient and coupled to the patient's spine, wherein the first piece includes a recessed portion; a first magnet coupled to the first piece; a second piece configured to be implanted into the patient and juxtaposed with the first piece, wherein the second piece includes a base portion surrounding a raised portion, wherein the raised portion is configured to be at least partially received within the recessed portion of the first piece so as to facilitate alignment of the first and second pieces; and a second magnet coupled to the second piece, wherein the first magnet exerts a desired magnetic force on the second magnet.

### RELATED APPLICATIONS

This application claims priority under 35 U.S.C. § 119(e) to U.S. provisional application Ser. No. 60/759,094, entitled “Magnetic Spinal Implants,” filed on Jan. 13, 2006, the entirety of which is incorporated by reference herein.

### BACKGROUND OF THE INVENTION

1. Field of the Invention

The present invention relates to spinal implants and, more particularly, to spinal implant devices utilizing magnetic fields to provide magnetic attraction and/or repelling forces between two or more vertebral bodies or spinal structures.

2. Description of the Related Art

Various types of spinal implant devices are well known in the art. For example, implants are commonly used to replace all or a portion of damaged vertebral bodies and intervertebral disks of the human spine. One disadvantage of conventional spinal implant devices, however, is that they are typically made of a biocompatible metal or other similar material which is hard and rigid and, therefore, do not provide any “cushion” or “shock absorption” to relieve axial loads or other forces exerted on the vertebral structures to which the implants are attached. This unnatural hardness and rigidity can cause considerable discomfort to patients and can sometimes damage the spinal bones that the implants are attached to. Additionally, such spinal implant devices significantly decrease the flexibility of the spine, causing discomfort to patients and sometimes damaging adjacent spinal structures which must compensate for the rigidity of the implants. These abnormal bio-mechanical properties also increase the rate of implant wear and subsequent failure. Conversely, spinal implants made of more elastic and compressible, non-metal materials are not as mechanically reliable as metal or other similarly rigid implants and after prolonged stress (e.g., repeated compression and decompression) can degrade or suffer from “wear and tear,” losing their structural integrity.

Furthermore, existing spinal implant devices do not provide a dynamic distraction force that increases as axial and/or compression forces are exerted on the spine. Additionally, conventional spinal implant devices do not provide a dynamic distraction force that increases as unwanted curvature of the spine (e.g., scoliosis, kyphosis, disk degeneration, etc.) begins to occur, or increases in severity, in order to provide a dynamic counteracting force against such unwanted curvatures of the spine.

Thus, what is needed is a spinal implant device capable of providing a dynamic cushion and shock absorption between two spinal structures. What is also needed is an implant device capable of providing attraction or repelling forces between two spinal structures in order to compensate for unwanted curvatures of the spine or abnormal forces exerted on the spine.

### SUMMARY OF THE INVENTION

The present invention addresses the above and other needs by providing magnetized or magnetically susceptible spinal implant devices configured to produce or utilize magnetic fields in order to provide desired attraction and/or repelling forces between two or more vertebral structures.

During the twentieth century, materials scientists and engineers developed stronger and stronger permanent magnets—alnico magnets in the 1930s, ferrite (ceramic) magnets in the 1950s, and rare-earth magnets in the 1970s and 1980s. The latest rare-earth magnets, such as neodymium-iron-boron, are more than one hundred times more powerful than the steel magnets available in the nineteenth century. It is estimated that rare-earth magnets, such as neodymium-iron-boron (hereinafter “neodymium”) and samarium-cobalt magnets have magnetic strength energies of up to 35 megagauss-oersted, which can lift up to an estimated 350 pounds and repel approximately two-thirds of this weight, or 230 pounds. Additionally, neodymium magnets are virtually permanent, losing only about 1% of their strength over a period of 10 years.

Electromagnets, which are also well known in the art, use a power source which provides current through a coil of wire typically wrapped around a paramagnetic or ferromagnetic core material. One main advantage of an electromagnet over a permanent magnet is that the magnetic field of an electromagnet can be rapidly turned on and off or manipulated over a wide range by controlling the magnitude and/or direction of electric current through the coil.

In one embodiment of the invention, an implant device configured to replace all or a portion (e.g., nucleus) of an intervertebral disk includes a first piece configured to be secured to a first vertebral body and a second piece configured to be secured to a second vertebral body adjacent the first vertebral body, wherein the first piece includes a first magnetic element and the second piece includes a second magnetic element and wherein the first magnetic element repels the second magnetic element to provide a repelling or distraction force between the first and second vertebral bodies. In a further embodiment, the first and second pieces are encapsulated in a biocompatible elastomer, polymer or fabric that holds the pieces together during implantation into a patient and limits or resists unwanted relative movement between the pieces after implantation.

In another embodiment, a magnetic implant system configured to provide repelling forces between two adjacent structures of a spine includes a first magnetic member configured to be secured to or embedded in a first spinal structure and a second magnetic member configured to be secured to or embedded in a second spinal structure, wherein the first and second magnetic members provide a repelling force between the first and second spinal structures.

In one embodiment, a magnetic spinal implant system includes a plurality of members configured to be secured and/or embedded in selected respective spinal structures, wherein the plurality of members provide repelling and/or attraction forces between each other so as to provide forces to counterbalance undesired curvature or curvature tendencies (e.g., hyphosis or scoliosis) of the spine or collapse due to trauma or degeneration.

In another embodiment, a magnetic spinal implant system utilizes a first member configured to be secured and/or embedded within a first spinal structure within a patient and a second member configured to be secured to and/or embedded within a second spinal structure of the patient, adjacent the first spinal structure, wherein at least the first member comprises an electromagnet, the spinal implant system further comprising a power source configured to be implanted within the patient for providing electric current to the electromagnet. In further embodiments, the magnetic spinal implant system also comprises an implantable radio transceiver and microcontroller coupled to the power source, wherein the transceiver is capable of communicating with an external device via radio telemetry and providing commands to the microcontroller from the external device, wherein the commands control the operation (e.g., on/off times, durations, current levels, etc.) of the power source and, hence, the spinal implant.

In another embodiment, a magnetic spinal implant includes first and second pieces that interlock with one another so as to provide a limited range of motion with respect to one another. The first and second pieces each include at least one magnetic element such that the first and second pieces magnetically repel or attract each other. In a further embodiment, counteracting magnetic forces are provided between the first and second pieces so as to provide “magnetic cushion” forces that resist both distraction and compression of the first and second pieces with respect to one another.

### BRIEF DESCRIPTION OF THE DRAWINGS

FIG. 1A illustrates a cross-sectional side view (sagital view) of two magnetic intervertebral disk implant devices, in accordance with one embodiment of the invention.

FIG. 1B illustrates a cross-sectional front view (coronal view) of the two magnetic intervertebral disk implants of FIG. 1A.

FIG. 2A illustrates a cross-sectional view of a magnetic implant, in accordance with one embodiment of the invention.

FIG. 2B illustrates a top view of one piece of the magnetic implant of FIG. 2A.

FIG. 3 illustrates a cross-sectional top view of one piece of the magnetic intervertebral disk replacement implant of FIGS. 1A and 1B, in accordance with one embodiment of the invention.

FIG. 4 illustrates a cross-sectional top view of one piece of the magnetic intervertebral disk nucleus replacement implant of FIGS. 1A and 1B, in accordance with one embodiment of the invention.

FIG. 5 illustrates a cross-sectional top view of one piece of a magnetic intervertebral disk implant, in accordance with one embodiment of the invention.

FIG. 6 illustrates a cross-sectional side view of a magnetic implant system configured to provide desired repelling and/or attraction forces between adjacent intervertebral bodies and interspinous processes, in accordance with one embodiment of the invention.

FIG. 7 illustrates a cross-sectional side view of a magnetic spacer configured to be coupled to two pedicle screws to provide desired repelling and/or attraction forces between adjacent vertebral bodies, in accordance with one embodiment of the invention.

FIG. 8 illustrates a cross-sectional view of the magnetic spacer of FIG. 7.

FIG. 9 illustrates a cross-sectional view of a multi-level magnetic spacer system, in accordance with one embodiment of the invention.

FIG. 10 illustrates a front view of two multi-stage magnetic spacers secured to respective vertebral bodies by means of vertebral body screws, in accordance with one embodiment of the invention.

FIG. 11 illustrates a cross-sectional view of a multi-level magnetic spacer system, in accordance with another embodiment of the invention.

FIG. 12 illustrates a side view of an expandable intervertebral disk replacement device, in accordance with one embodiment of the invention.

FIG. 13 illustrates a side view of a magnetized expandable vertebral replacement or disk replacement device, in accordance with one embodiment of the invention.

FIG. 14 illustrates a cross-sectional view of a magnetic intervertebral disk replacement device, including electromagnets, in accordance with one embodiment of the invention.

FIG. 15 illustrates a block diagram of a power module for controlling an electromagnet of a spinal implant device, in accordance with one embodiment of the invention.

FIG. 16A is a cross-sectional side view of a magnetic spinal implant, in accordance with one embodiment of the invention.

FIG. 16B is a cross-sectional top view of the magnetic spinal implant of FIG. 16A, taken along lines A-A of FIG. 16A.

FIG. 17A is a cross-sectional side view of a magnetic spinal implant, in accordance with another embodiment of the invention.

FIG. 17B is a perspective view of the top piece of the magnetic spinal implant of FIG. 17A.

FIG. 17C is a perspective view of the bottom piece of the magnetic spinal implant of FIG. 17A.

FIG. 18 is a cross-sectional side view of a magnetic spinal implant, in accordance with another embodiment of the invention.

FIG. 19A is a cross-sectional side view of a three-piece expandable magnetic spinal implant, in accordance with another embodiment of the invention.

FIG. 19B is a top view of magnetic spinal implant of FIG. 19A.

FIG. 20 is a cross-sectional side view of a magnetic spinal implant, in accordance with another embodiment of the invention.

### DETAILED DESCRIPTION OF THE PREFERRED EMBODIMENTS

The invention is described in detail below with reference to the figures, wherein like elements are referenced with like numerals throughout. It should be understood that the figures are not necessarily drawn to scale. Rather, they are intended to show certain features and elements of various exemplary embodiments of the invention.

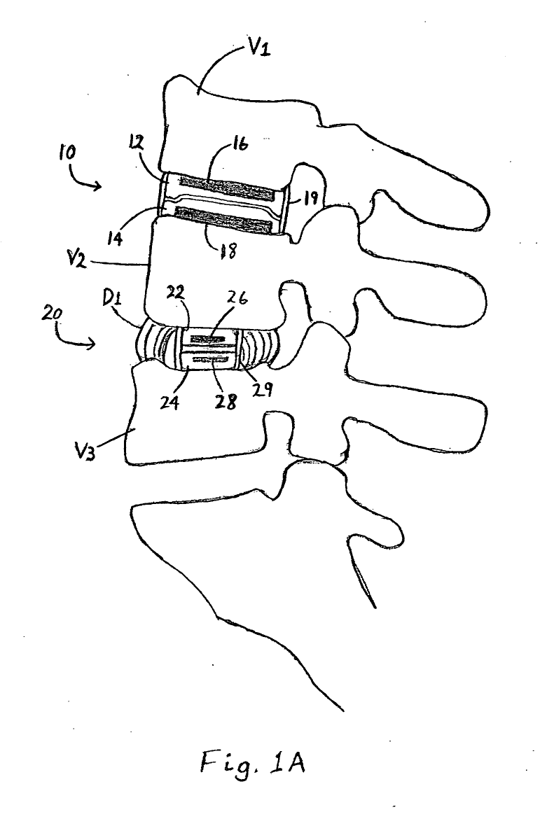

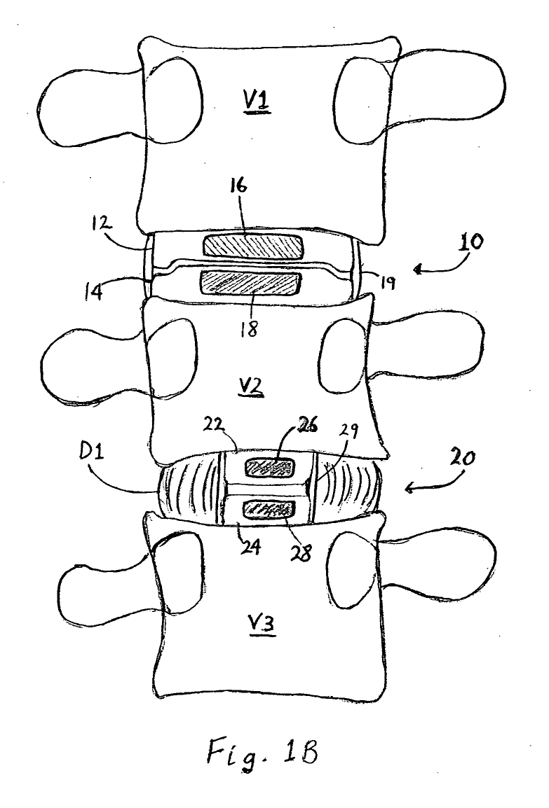

FIG. 1A illustrates a cross-sectional side view of a patient's spine having two magnetic intervertebral disk implants 10 and 20 implanted therein. The implants 10 and 20 are illustrated together for purposes of discussion only and need not be used together in actual applications. The magnetic implant 10 is designed to replace the entire disk between vertebras V1 ad V2 and includes a first piece 12 configured to be secured to first vertebral body V1 and a second piece 14 configured to be secured to second vertebral body V2. Each of the first and second pieces 12 and 14 can be secured to their respective vertebral bodies using known means and techniques. For example, the surfaces of the first and second pieces 12 and 14 that contact their respective vertebras can be rough and porous to encourage new tissue and bone from the vertebras to grow into the implant surfaces, holding them securely in place. This tissue ingrowth provides a reliable bond between the implant pieces 12 and 14 and their respective vertebras V1 and V2. Additionally, each implant piece 12 and 14 can include anchoring spikes, screws and/or other protrusions (not shown) at their respective interface surfaces, which are designed to be embedded and lodged into the bone of the vertebral bodies V1 and V2 for added fixation. Other known securing means and techniques may also be used, such as bone cement and/or fixation screws. Furthermore, each piece 12 and 14, including their respective magnets 16 and 18, may have holes or other windows (not shown) formed therein for insertion of bone growth material or otherwise promoting bone and/or tissue growth therethrough.

In one embodiment, the magnets 16 and 18 magnetically repel each other so as to provide a desired repelling force between vertebras V1 and V2. Various types and sizes of magnets may be used for magnets 16 and 18 depending on the desired level of repelling force and the duration of magnetic repulsion desired. In one embodiment, the magnets 16 and 18 comprise neodymium magnets. As mentioned above, it is estimated that neodymium magnets will lose only about 1% of their magnetic power over a period of ten years. In a further embodiment, the magnets 16 and 18 may be encased or embedded in any suitable biocompatible material or combination of materials including, e.g., PEEK, polyethylene, titanium, titanium alloy, carbon graphite, ceramic etc. In alternative embodiments, each implant piece 12 and 14 may be made entirely from a permanent magnet or a magnetic alloy material or composition.

As discussed in further detail below, in other embodiments, one or both of the magnets 16 and 18 may be an electromagnet coupled to an implantable power source, which controls current supplied to the electromagnet. In alternative embodiments, one of the magnets 16 or 18 may be a permanent magnet or an electromagnet while the other magnet 16 or 18 may simply be a magnetically susceptible metal or metal alloy.

The magnetic repulsion provided by magnets 16 and 18 provides a dynamic distraction force between vertebras V1 and V2. As is known in the art, magnets having the same polarity repel each other and this repelling force increases exponentially in inverse proportion to the distance between the magnets. Thus, as axial loads are exerted onto a spine and vertebras V1 and V2 are compressed and pushed closer together, the repulsion force provided by the magnets 16 and 18 dynamically increases as the magnets are moved closer together, thereby dynamically damping the force exerted on the vertebras V1 and V2. The amount of repulsion force provided by the magnets 16 and 18 can be tailored for each application and the particular needs of each patient. Various types, sizes, shapes of permanent magnets and magnetic alloys exhibit different magnetic attraction and repulsion forces. Additionally, the magnets 16 and 18 can be encased or embedded in various biocompatible materials (e.g., titanium, titanium alloys, ceramics, polymers, etc.) to provide various levels of magnetic shielding to further control the magnetic forces exerted between the magnets 16 and 18. Alternatively, the magnets 16 and/or 18 can comprise electromagnets to control the amount of distraction force between the magnets 16 and 18 by controlling the electric current applied to the electromagnets.

As shown in FIG. 1A, the first implant 10 can optionally include a cover or encasing 19 that partially or completely encases the first and second pieces 12 and 14. One purpose of the cover 19 is to prevent or resist undesired lateral and/or twisting motion between the first and second pieces 12 and 14 which may occur as a result of the magnetic forces produced by 16 and 18 or by other mechanical forces during movement of the V1 and V2 vertebras relative to each other. Another purpose of the cover or encasing is to hold the pieces together during implantation and subsequently resist lateral and/or twisting motion, for example, of the first and second pieces 12 and 14 after implantation. Depending on the strength of the repelling forces generated by the magnets 16 and 18, the cover 19 should be designed and made from a suitable material to counteract the undesired lateral and or twisting forces, for example. In various embodiments, the cover 19 can be made from any suitable biocompatible elastomer, polymer, resin, fabric or combination of these materials and compositions. In another embodiment, a further purpose of the cover 19 can be to shield the magnets 16 and 18 from external magnetic or electromagnetic forces. In this embodiment, the cover 19 can be made from any suitable magnetic or electromagnetic shielding material.

As shown in FIG. 1A, the second magnetic implant 20 is smaller than the first implant 10 and designed to replace only a portion (e.g., nucleus) of the intervertebral disk D1. Similar to the first implant 10, the implant 20 includes a first piece 22 designed to be coupled or secured to vertebra V2 and a second piece 24 designed to be coupled or secured to vertebra V3. The first and second pieces 22 and 24 may be coupled or secured to their respective vertebra using any known means. The first and second pieces 22 and 24 each include respective magnets 26 and 28 for providing a dynamic distraction force between vertebras V2 and V3, as discussed above with respect to the first implant device 10. The second implant 20 further includes a cover 29 that may be similar to the cover 19 of implant 10, discussed above. The implants 10 and 20 may be surgically implanted using any method, including minimally invasive techniques which are well known in the art.

FIG. 1B illustrates a cross-sectional front view of the implants 10 and 20. In one embodiment, the implant pieces 12, 14, 22 and 24 are circular or oval shaped disks. However, any desired shape may be utilized in accordance with the invention. Implant 20 is designed to replace the nucleus (a.k.a., “inner goo”) of the intervertebral disk and its shape corresponds to the shape of the space occupied by the nucleus within the tough containment ring or annulus of the intervertebral disk D1.

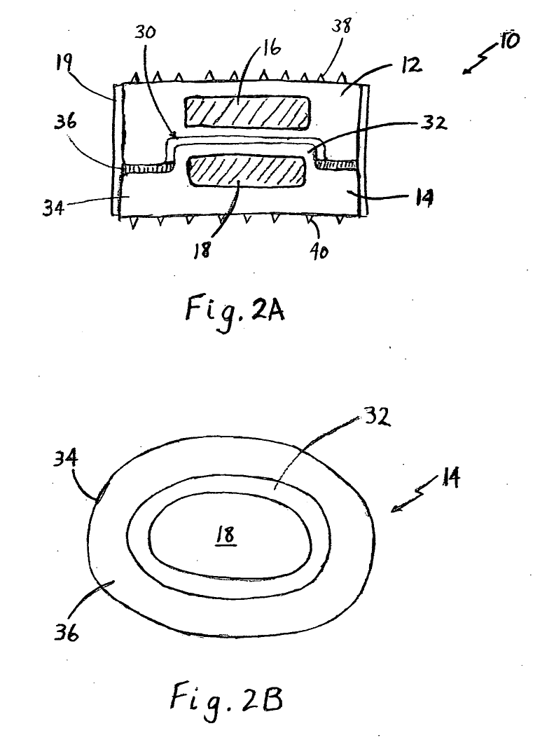

FIG. 2A illustrates a cross-sectional side view one exemplary embodiment of the implant 10 of FIGS. 1A and 1B. The first piece 12 includes a recessed or concave center portion 30 configured to receive a raised central island portion 32 of the second piece 14 that rises above a surrounding base portion 34 of the second piece 14. Thus, the recess 30 of the first piece 12 and the raised portion 32 of the second piece 14 facilitate alignment of the first and second pieces 12 and 14 with respect to one another. Additionally, as shown in FIG. 2A, the implant device 10 may include an optional gasket 36 that surrounds the island portion 32 of the second piece 14. In one embodiment, this gasket 36 may be made from an elastic or compressible biocompatible material to provide an additional physical cushion between the first and second pieces 12 and 14, respectively.

In a further embodiment, the implant 10 is laterally encased with a cover or wrap 19 which prevents or resists undesired lateral movement between the first and second pieces 12 and 14, as discussed above. In one embodiment, the cover 19 may be made from the same material as gasket 36 and integrally formed therewith. In one embodiment, the cover 19 completely surrounds the implant 10 and provides a hermetically sealed device. Furthermore, the cover 19 can be used to seal the space between the first and second pieces 12 and 14 so that particles, debris and/or other physiological substances can be shielded. Although the magnets 16 and 18 will typically provide sufficient repelling force to prevent the first piece 12 from making contact or exerting significant prolonged force onto the second piece 14, the gasket 36 can provide an additional secondary cushion between the first and second pieces 12 and 14. Additionally, the magnetic repelling forces will counteract the compression forces between the first and second pieces 12 and 14, thereby decreasing the wear rate of the gasket 36. A plurality of spikes or anchors 38 are placed on the top surface of the first piece 12 to facilitate securing the first piece 12 to the first vertebra V1. A plurality of spikes or anchors 40 are optionally placed on the top surface of the second piece 14 to facilitate securing the second piece 14 to the second vertebra V2.

FIG. 2B illustrates a top view of the second piece 14 which is configured generally in the shape of an oval, in accordance with one embodiment. The magnet 18 is located at the center of the oval and is similarly shaped. However, in other embodiments, the magnet 18 can be any other shape or configuration, including fenestrated, or ring-shape to accomplish a desired magnetic effect. The island 32 is also oval shaped and rises above the base portion 34, which surrounds the island 32. The gasket 36 is configured in the shape of an oval ring that surrounds the island 32 and rests on the top surface of the base 34 that surrounds the island 32. In preferred embodiments, the gasket 36 is made from any durable and compressible biocompatible elastomer, polyurethane or silicone, or other biocompatible materials, which are well known in the art.

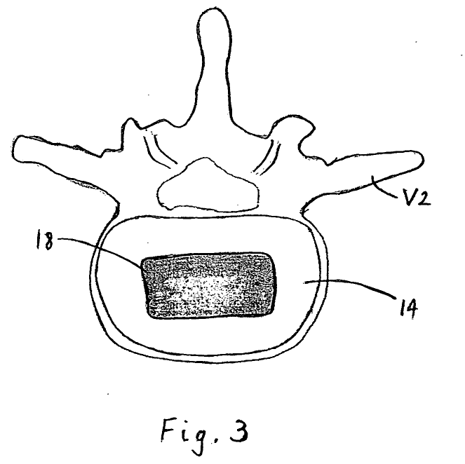

FIG. 3 illustrates another cross-sectional top view of the second piece 14 of the implant device 10 of FIGS. 1A and 1B, in accordance with one embodiment of the invention. The second piece is formed in the shape of a typical intervertebral disk and the magnet 18 is similarly shaped and located at a substantially central location of the second piece 14. In one embodiment, the second piece 14 is made from a biocompatible metal such as titanium and the magnet 18 is a neodymium magnet that is embedded, formed or encased in the titanium body of the second piece 14. However, other types of biocompatible metals or materials may be used. For example, the second piece 14 may comprise a titanium alloy, ceramic, polymer, or carbon graphite material that partially surrounds or completely encases the magnet 18. In one preferred embodiment, the second piece 14 comprises titanium and/or ceramic materials which are substantially transparent to magnetic fields and thus will not significantly interfere with the magnetic forces exerted by the magnet 18. As shown in FIG. 3, if the magnet 18 is located at a substantially central location within the second piece 14, the magnetic forces exerted by the magnet 18 is substantially balanced at the center of the second piece 14 of the implant 10. However, in alternative embodiments, the magnet 18 can be located off-center so as to provide a magnetic repelling or attraction force at a desired off-center axis of the vertebra to which it is attached.

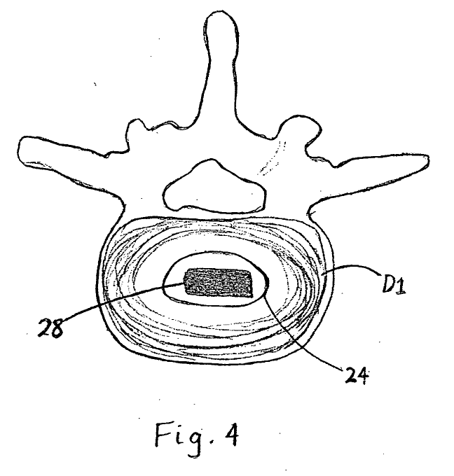

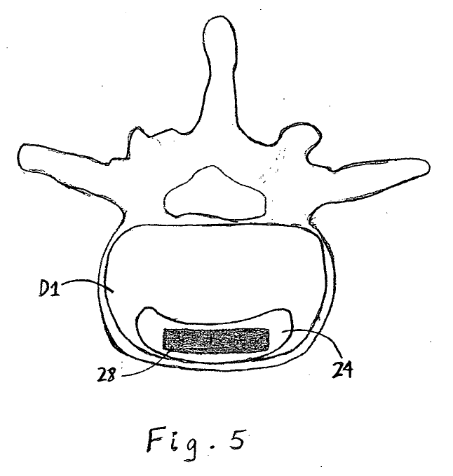

FIG. 4 illustrates a cross-sectional top view of the second piece 24 of the disk nucleus replacement implant device 20 of FIGS. 1A and 1B. The implant 20 is configured to replace the nucleus of an intervertebral disk. After the damaged nucleus (not shown) is removed, the first and second pieces 22 and 24 are inserted so that they are contained within the outer containment ring 30 of the disk D1. As shown in FIG. 4, the second piece 24 is located at a center portion of the disk D1. The first piece 22 would be located at a corresponding center portion on a vertebra (e.g., V2) above the second piece 24. The second piece 24 includes a magnet 28 located therein to provide a dynamic repelling force against a corresponding magnet 26 of the first piece 22 (FIGS. 1A and 1B). As discussed above, the first and second pieces 22, 24 may be made from any one or combination of biocompatible materials (e.g., titanium, titanium alloy, ceramic, PEEK, PEEKEK, nitinol, carbon graphite, polymer, etc.), which partially or completely encases the magnets 26 and 28, respectively. Furthermore, pieces 22 and 24 can be contained within a cover (e.g., a biocompatible polymer or elastomeric cover), which can be implanted into the patient as single device. In one embodiment, the cover provides a hermetically sealed implant 20.

FIG. 5 illustrates an alternative embodiment of a second piece 24 and its corresponding magnet 28 of the partial disk replacement implant 20. In this embodiment, the first piece 22 (shown FIGS. 1A and 1B) and the second piece 24, respectively, do not replace the nucleus but, rather, are configured to be secured to their respective vertebras V2 and V3 (FIGS. 1A and 1B) at an anterior off-center location of the vertebras. Thus, in this embodiment, a portion of the containment ring 30 is removed and the second piece 24 is positioned and secured to an anterior region (i.e., toward the front of the patient) of the vertebra V3. The first piece 22 (not shown in FIG. 5) is positioned and secured to a corresponding anterior region of the vertebra V2 above the second piece 24. In this position, the magnets 26 and 28 of the first and second pieces 22 and 24, respectively, will provide a dynamic distraction force at an off-center anterior region of the spine. For example, if the patient bends forward so that the anterior portions of the vertebras come closer together, the magnets 26 and 28 will provide a dynamic repelling or distraction force so as to cushion and/or resist the forward bending motion. Additionally, the magnets 26 and 28 will resist or compensate for unwanted curvature of the spine in the anterior, or forward-bending, direction.

In alternative embodiments, the magnets 16 and 18, or 26 and 28, for example, may be located at any position (e.g., center, posterior, anterior, lateral) within their respective first and second pieces 12, 14, 22 and 24, so as to provide desired repelling, distraction and/or cushioning forces along one or more axes of the spine. Additionally, the pieces 12 and 14 and/or 22 and 24, and their respective magnets 16, 18, 26 and 28 may be any desired size, configuration, shape and/or magnetic power so as to achieve desired magnetic forces between two or more vertebral bodies (e.g., V1, V2 and V3). For example, as is known in the art, different regions of the spine (e.g., lumbar and thoracic) have a tendency to curve in different directions. Thus, in various embodiments of the invention, the location, size, configuration, shape and/or magnetic power of the pieces 12, 14, 22 and 24 and their respective magnets 16, 18, 26 and 28 may be designed to compensate for, or at least deter, unwanted curvature of one or more regions of the spine. For example, the magnetic implants 10 and/or 20 may be used to deter or correct unwanted curvature of the spine that occurs as a result of scoliosis, osteoporosis or kyphosis.

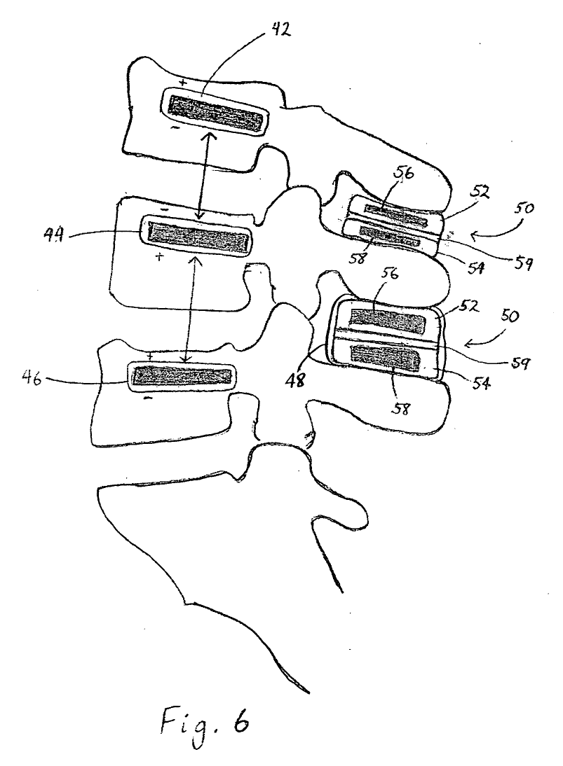

The invention is not limited to implants that partially or completely replace intervertebral disks, as described above. As shown in FIG. 6, magnetic implants 42, 44 and 46 may be configured to be implanted or embedded within respective vertebral bodies or vertebras V1, V2 and/or V3. The implants 42, 44 and 46 can be implanted in any desired location within the vertebras and designed to provide desired repelling and/or attraction forces between adjacent implants so as to provide desired on-center and/or off-center magnetic forces along one or more axes of the spine. As discussed above, the location, size, configuration, shape and/or magnetic power of the implants 42, 44 and 46 may be designed to compensate for and/or deter unwanted curvatures or weakening of the spine due to various spinal ailments. In one embodiment, the implants 42, 44 and/or 46 comprise neodymium magnets partially or completely encased within respective titanium housings or casings. However, other types of magnets and biocompatible materials for the housing may be used. Additionally, as explained in further detail below, in further embodiments the implants may comprise electromagnets coupled to a power module for providing and controlling current provided to the electromagnets.

As further illustrated in FIG. 6, in additional embodiments of the invention, one or more magnetic implants 50 are configured to be placed and secured between respective interspinous processes or lamina. The implants 50 are similar to the implant 10 described above with respect to the FIGS. 1A and 1B but are configured to be positioned and secured to respective interspinous processes and/or laminas, instead of replacing an intervertebral disk D1. Each implant 50 includes first and second pieces 52 and 54, respectively, configured to be secured to respective interspinous processes, utilizing any one of various known techniques and means, including press-fit, screws, hooks, blades, stops, etc. The first and second pieces 52 and 54 each include respective magnets 56 and 58 for providing desired magnetic forces with respect to one another. The implant 50 further includes an optional compressible gasket 59 made from any suitable biocompatible material for providing an additional cushion between the first and second pieces 52 and 54. As discussed above, the location, size, configuration, shape and/or power of the first and second magnets 56 and 58 may be adjusted and designed to achieve desired repelling and/or attraction forces in one or more directions along the spine. In one embodiment, the implant device 50 includes a housing or cover 48 that is designed to hold the pieces 52 and 54 together and prevent lateral shifting of the pieces. The cover 48 of the implant 50 may be made from any one or combination of materials described above. In a further embodiment, the cover 48 is designed to substantially shield the magnets from external magnetic forces while allowing magnetic forces directly between the adjacent magnets 56 and 58. The cover 48 may be made from any suitable biocompatible material or combination of materials that can function as a magnetic shield. In one embodiment, the magnets 56 and 58 comprise electromagnets.

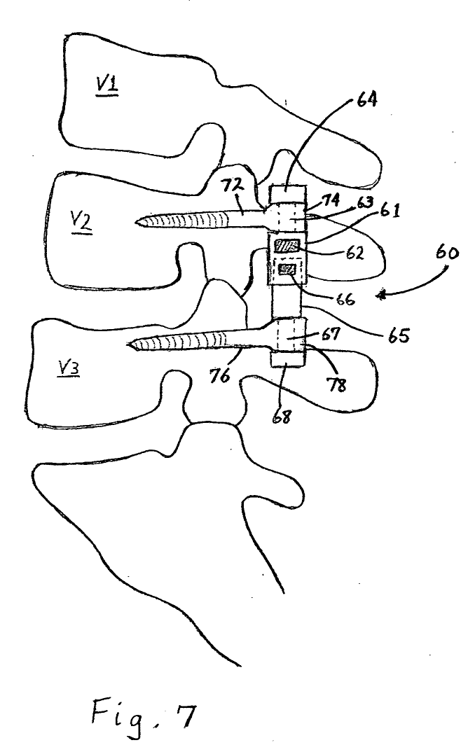

Referring to FIG. 7, in accordance with additional embodiments of the invention, a magnetic implant 60 includes a first piece 61 having a first magnet 62 therein and a second piece 65, configured to be coupled to the first piece 62 and having a second magnet 66 therein. The first piece 61 is configured to be secured to a first vertebral body by a first pedicle screw 72 and the second piece 65 is configured to be secured to a second vertebral body by a second pedicle screw 76. The first piece 61 has a narrow portion 63 configured to be received within a coupling or locking head 74 of the first pedicle screw 72. The second piece 65 also has a narrow portion 67 configured to be received within a coupling or locking head 78 of the second pedicle screw 76. In one embodiment, each of the first and second pieces 61 and 65 has respective end portions 64 and 68 that are configured to assist with holding the first and second pieces 61 and 65 within the locking heads 74 and 78 of the first and second pedicle screws 72 and 76, respectively. The pedicle screws 72 and 76 and their respective locking heads 74 and 78 may be any one of various pedicle screws that are well known in the art. Various locking heads 74 and 78 and locking mechanisms are also known in the art. The invention may utilize any of these pedicle screws and their corresponding locking heads.

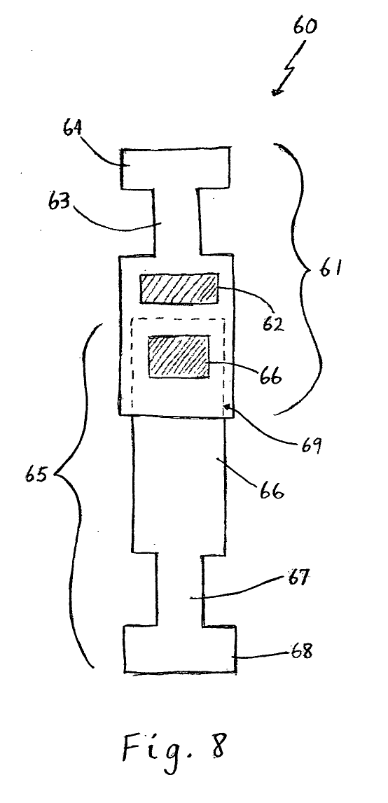

FIG. 8 illustrates a perspective view of the magnetic implant 60 of FIG. 7 without the pedicle screws 72 and 76. In one embodiment, the first piece 61 has a cylindrical shaped recess or chamber 69 within its main body configured to receive at least a portion of the second piece 65 therein as indicated by the dashed lines. The second piece 66 has a corresponding cylindrically shaped portion 66 that is sized and configured to be snugly but slidingly received within the cylindrical chamber 69 so as to be able to move up and down within the chamber 69, similar to a piston within a piston cylinder. In one embodiment, the second piece 65 and engagement portion 66 are made from a solid biocompatible metal material (e.g., titanium) so as to provide increased strength and durability. The magnet 66 is embedded within an end portion of the engagement/piston portion 66. As axial loads are exerted on a patient's spine (not shown), the first piece 61 is compressed into the second piece 65 and the first and second magnets 62 and 66 exert a magnetic repulsion force against each other that dynamically increases as the magnets 62 and 66 are brought closer together. In one embodiment, the first and second pieces 61 and 65 are made from titanium and the magnets 62 and 66 are neodymium magnets. In alternative embodiments the magnets 62 and 66 are electromagnets. In a further embodiment, the interior surface of the chamber 69 is lined with a suitable polymer or teflon material so as to provide substantially frictionless movement of the engagement portion 66 within the chamber 69. As shown in FIG. 8, the first piece 61 includes a narrow portion 63, between the main body of the first piece 62 and an end portion 64, wherein the narrow portion 63 is configured to be received within a locking head of a first pedicle screw (FIG. 7) or other securing means. Similarly, the second piece 65 includes a narrow portion 67, between its main body and an end portion 68, wherein the narrow portion 68 is configured to be received within a locking head of a second pedicle screw 76 (FIG. 7) or other securing means.

It is understood that the above-described configurations of the first and second pieces are exemplary only and that other configurations may be utilized in accordance with the invention. For example, the narrow portions 63, 67, 64 and 68 of the first and second pieces 61 and 65, respectively, may or may not be present, or present in any desired combination depending on desired mechanical constructions and coupling techniques with various securing mechanisms (e.g., vertebral body and/or pedicle screws).

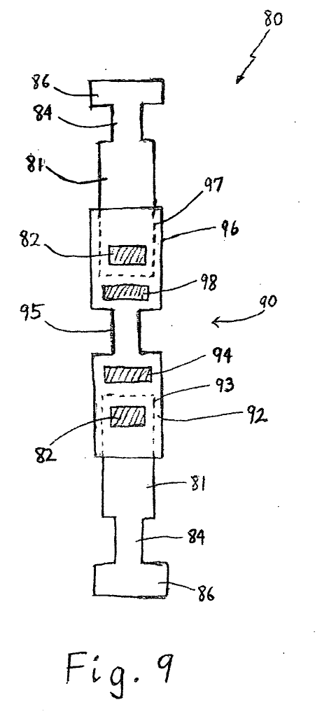

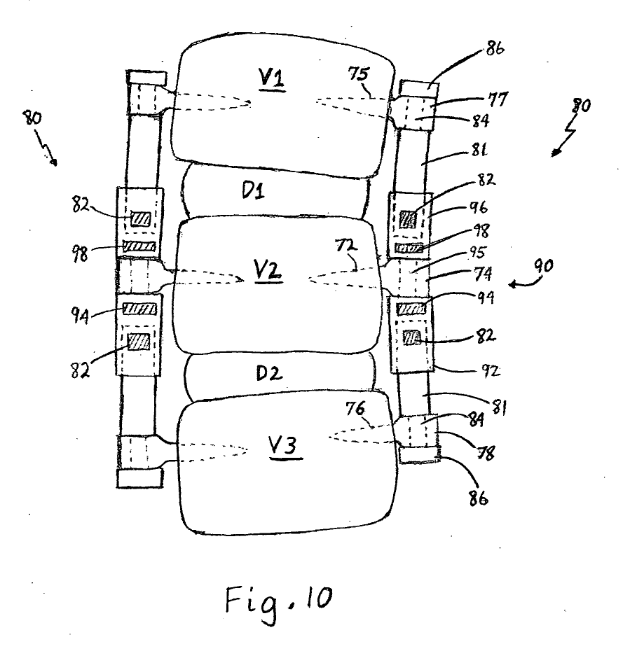

FIG. 9 illustrates a cross-sectional view of a multi-stage magnetic implant 80, in accordance with another embodiment of the invention. The implant 80 is similar to the implant 60 of FIG. 8 but is designed to be secured to three adjacent vertebral bodies (V1, V2 and V3), as shown in FIG. 10. The multi-stage implant 80 includes two end pieces 81 having respective magnets 82 therein, each of which are similar to the second piece 65 described above with respect to FIG. 8. Each of the end pieces 81 have a narrow portion 84, between their respective main bodies and end portions 86, the narrow portions 84 being configured to be coupled to and secured within respective pedicle screw heads. The multi-stage implant 80 further includes a center piece 90 having two opposing cylindrically shaped bodies 92 and 96 located on opposing sides of a narrow portion 95, wherein the narrow portion 95 is configured to be coupled to a third pedicle screw head. Each of the bodies 92 and 96 have respective cylindrically shaped recesses or chambers 93 and 97 for receiving at least a portion of the main body portions of respective end pieces 81. As discussed above, in one embodiment, the engagement/piston portions of the end pieces 81 are also cylindrically shaped so as to slidably fit inside the chambers 93 and 97.

FIG. 10 illustrates two multi-stage magnetic implants 80 (FIG. 9) secured to multiple vertebral bodies (V1-V3) on opposite sides of the vertebra, in accordance with one embodiment of the invention. Each implant 80 includes a center piece 90 having a narrow portion 95 coupled to the locking head 74 of a first screw 72, which is in turn secured to a middle vertebral body V2. Each top end piece 81 of each implant 80 is secured to a top vertebral body V1 by means of a screw 75 having a locking head 77 for receiving a narrow portion 84 of the top end piece 81. Similarly, each bottom end piece 81 is secured to a bottom vertebral body V3 via a third pedicle screw 76 which receives the narrow portion 84 of the bottom end piece 81 within its locking head 78. In some embodiments, screws 72, 75 and 76 can be either pedicle screws or vertebral body screws. Thus, they can be used posteriorly (pedicle screws) or anteriorly (vertebral body screws) or any combination thereof. Additionally, other types of securing means known in the art may be utilized in accordance with the present invention.

The center piece 90 includes two magnets 94 and 98 located within respective bodies 92 and 96. Each of the end pieces 81 includes a respective magnet 82 which provides a repelling force against respective magnets 94 and 98 located in the center piece 90. Thus, as axial forces are exerted onto a patient's spine, the end pieces 81 are compressed into the center piece 90 causing the bottom magnet 82 to move closer to magnet 94 and the top magnet 82 to move closer to magnet 98. As the magnets move closer together, the magnetic repulsion forces between them increase to provide a cushioning or dampening force against the axial forces. The corresponding magnets 82′, 94′ and 98′ located in the multi-stage implant 80 located on the other side of the vertebras behave in similar fashion.

As shown in FIG. 10, one or more multi-stage implantable devices 80 can be utilized to compensate for or prevent unwanted curvatures of the spine due to various spinal ailments (e.g., scoliosis, osteoporisis, etc.). By implementing magnetized implants as described herein at strategic locations of the spine, magnetic forces can be used to counteract or prevent such unwanted curvatures of the spine. It is understood, however, that in alternative embodiments, only one implant 80 may be attached to one side of the spine or to any suitable position (e.g., posterior, anterior and/or lateral surfaces) on the spine. Additionally, in other embodiments, two or more implants 80 may be secured to the spine to provide increased support and “shock-absorbing” functionality. Furthermore, one of ordinary skill in the art would readily recognize that the device 80 can be designed to span a larger number of vertebras than that illustrated in FIGS. 9 and 10. Additionally, various configurations of the device 80 may be utilized in accordance with the invention. For example, the “female” configuration of center piece 90 may be changed to a “male” configuration and the end pieces 81 can correspondingly be changed to have a “female” configuration for coupling with the center piece 90.

FIG. 11 illustrates an alternative embodiment of a multi-stage magnetic spacer system 80′ in accordance with the present invention. The multi-stage spacer system 80′ is similar to the system 80 illustrated in FIG. 9 except that it is configured to be secured to four adjacent vertebral bodies. The system 80′ includes a first end piece 81 that is identical to the end piece 81 of FIG. 9. The system 80′ further includes two exemplary middle pieces 90′ that have a first body portion 92 that is identical to the body portion 92 of FIG. 9. However, on the other side of respective narrow coupling portions 95, each of the middle pieces 90′ includes a male-configuration body portion 94′, instead of the female-configuration body portion 94 of FIG. 9. A modified end piece 81′ has a female configuration to receive the male-configuration body portion 94′ of the top-most middle piece 90′. As would be readily apparent to one of skill in the art, the multi-stage spacer system 80′ illustrated in FIG. 11 can easily be configured to be secured to any number of two or more adjacent vertebras by simply inserting the desired number (0 to N) of middle pieces 90′. Each of the pieces 81, 81′ and 90′ have respective magnet elements 82, 82′ and 94′ and 98′ that function in similar fashion as the magnet elements discussed above with respect to FIG. 9.

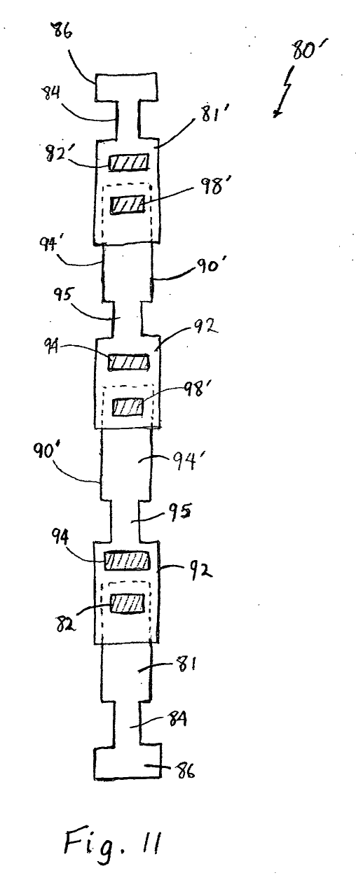

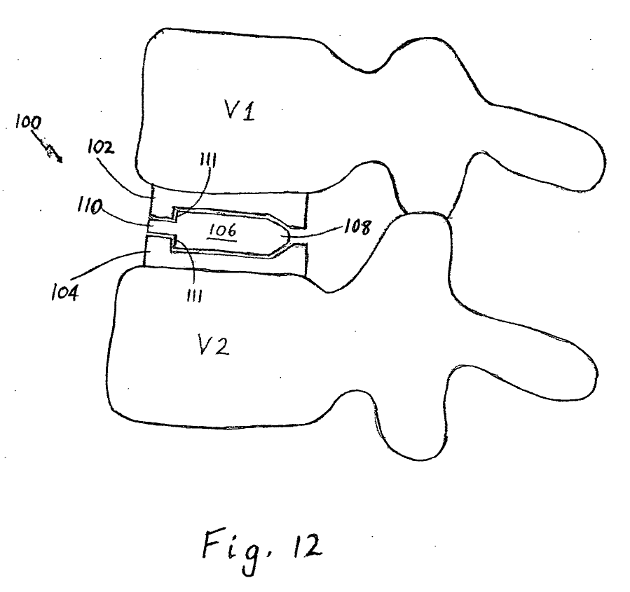

FIG. 12 illustrates one embodiment of an intervertebral disk replacement device 100 that includes an interchangeable spacer similar to that described in currently pending and commonly-owned U.S. application Ser. No. 11/508,003 entitled “Expandable Implant Device With Interchangeable Spacer,” filed Aug. 22, 2006, the entirety of which is incorporated by reference herein (hereinafter referred to as “the '003 application”). The expandable disk replacement device 100 includes a first piece 102 configured to be attached and secured to a first vertebral body V1 and a second piece 104 configured to be attached and secured to a second vertebral body V2. An interchangeable spacer 106 is configured to be inserted between the first and second pieces 102 and 104, respectively. Each of the first and second pieces 102 and 104, respectively, and the interchangeable spacer 106 can be surgically implanted in the same manner using the same or similar tools and techniques described in the '003 application. By selecting an interchangeable spacer 106 having desired dimensions, the overall height of the implant device 100 can be custom tailored to fit the needs of a particular patient and/or achieve a desired amount of distraction between the first and second vertebral bodies V1 and V2. As shown in FIG. 12, the spacer 106 has a tapered leading edge 108 for easy insertion into the space between the first and second pieces 102 and 104. The rear of the spacer includes a narrow base portion 110 from which two backstop walls 111 step up to form a wider body portion of the spacer 106. The backstop walls 111 interlock with corresponding walls within the internal cavity or recess formed between the first and second pieces 102 and 104, respectively, to prevent the spacer 106 from backing out of the cavity. The first and second pieces 102 and 104, and the spacer 106 may be made from any desired biocompatible material and can be secured and placed into their respective positions using any of the techniques described in the '003 application, or any other technique that is known or readily apparent to those of skill in the art. In one embodiment, the first and second pieces 102 and 104, and the spacer 106, have insertion holes (not shown) similar to those described in the '003 application for coupling to respective insertion tools for facilitating implantation of the first and second pieces 102 and 104, and the interchangeable spacer 106, as also described in the '003 application.

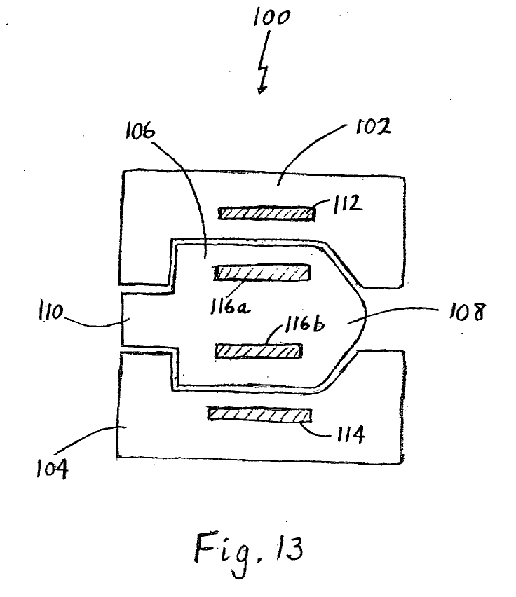

FIG. 13 shows a cross sectional view of the implantable intervertebral disk replacement device 100 of FIG. 12. It is understood that all the figures herein are not necessarily drawn to scale. Therefore, FIG. 13 can also represent an expandable vertebral body replacement device, for example, as well as a disk replacement device. The inventive concepts discussed herein may be equally applied to either type of device since one device is simply a smaller version of the other. As shown in FIG. 13, the first piece 102 includes a first magnet 112 and the second piece 104 includes a second magnet 114. The interchangeable spacer 106 includes two magnets 116a and 116b wherein the magnet 116a is configured to induce a magnetic force between itself and the first magnet 112 and the magnet 116b is configured to induce a magnetic force between itself and the second magnet 114. In alternative embodiments, the magnets 116a and 116b can be a single integral magnet 116 instead of two separate magnets. The polarity of the magnets 112, 114, 116a and 116b can be selected such that the magnetic forces induced between magnets 116a and 112 and between magnets 116b and 114 can both be repelling forces, one repelling force and one attraction force, or both attraction forces. If both are repelling forces, this configuration provides a maximum level of distraction and shock absorption against axial compression forces. If one is attraction and the other is repulsion, this configuration provides an intermediate level of distraction while magnetically holding the spacer 106 against either the first or second piece 102 or 104, respectively. If both provide attraction forces, the interchangeable spacer 106 is magnetically attached and held to each of the first and second pieces 102 and 104 to provide maximum holding strength between the spacer 106 and each of the first and second pieces 102 and 104.

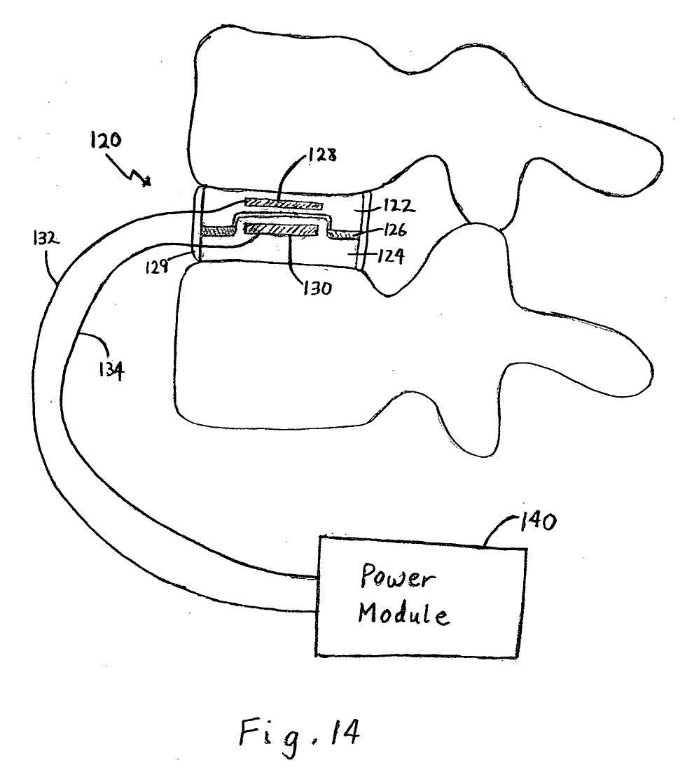

FIG. 14 illustrates a cross-sectional side view of a magnetic implant device 120 comprising two electromagnets 128 and 130, in accordance with one embodiment of the invention. The implant device 120 is similar to the implant device 10 of FIGS. 1A, 1B, 2A and 2B except that the magnets 128 and 130 are electromagnets. It is understood that any of the magnets described above may be implemented as electromagnets as described herein with respect to FIG. 14. The magnetic implant device 120 includes a first piece 122 configured to be attached to a surface of a first vertebra V1 and a second piece 124 configured to be attached to a second vertebra V2. An optional gasket ring 126 is positioned between the first and second pieces 122 and 124 similar to that described above with respect to FIGS. 2A and 2B. Additionally, the implant device 120 includes a housing or wrapping 129 that partially or completely encases the implant device 120, in similar fashion as the cover 19 described above with respect to FIGS. 1A and 2A.

The first piece 122 includes the first electromagnet 128 that is partially or completely encased within the first piece 122. The first electromagnet 128 is coupled to a power module 140 via corresponding lead wires 132. Similarly, the second piece 124 includes a second electromagnet 130 partially or completely encased within the second piece 124 and coupled to the power module 140 via a second set of corresponding lead wires 134. The first and second pieces 122 and 124 may be made from any biocompatible material and, in one embodiment, is made from titanium or titanium alloy. The electromagnets 128 and 130 are embedded or encased within their corresponding first and second pieces 122 and 124, respectively, using any known techniques. For example, the magnets 128 and 130 may be placed into and bonded within cavities formed within their respective first and second pieces 122 and 124 using biocompatible cement, for example. Alternatively, the first and second magnets may be placed into the cavities and subsequently sealed within the cavities by pouring or injecting a biocompatible elastomer, polymer or resin into the cavity. Or, the first and second magnets 128 and 130 may be placed into their respective cavities, which are thereafter sealed by welding metal lids to the openings of the cavities. The lids can have appropriate holes or grooves to allow lead wires 132 and 134 to emerge therethrough. Alternatively, if the first and second pieces 122 and 124 are made from polymer or resin material, for example, or other formable or moldable material, the electromagnets 128 and 130 may be encased or embedded within the first and second pieces 122 using forming, encapsulation and/or molding techniques known in the art (e.g., injection molding). All the magnetic elements described herein may be embedded or encased within their respective implant pieces using one or more of the techniques described above.

The power module 140 may be designed to be fully implanted subcutaneously at an advantageous location within a patient or configured to be attached to the skin of the patient with the lead wires 132 and 134 extending transcutaneously through the patient's skin and providing electrical contact with the fully implanted electromagnets 128 and 130 and an external power module 140. In one embodiment, the power module 140 is configured to be fully implanted subcutaneously and communicates with external devices via radio frequency (RF) telemetry. Various types of implantable RF telemetry devices are known in the art, for example, to monitor a patient's heart rate or condition, glucose monitoring, etc. Similar types of RF telemetry systems can be utilized in accordance with the present invention to control the power supplied to the electromagnets 128 and 130. As discussed in further detail below, the power module 140 includes a power source (e.g., a battery) and additional circuitry for controlling the amount, duration and/or intervals in which current is supplied to each of the electromagnets 128 and 130. In one embodiment, the power source and the techniques for delivering power to the electromagnets 128 and 130 may be similar to the power sources and techniques used to deliver power to inter-cardio defibrillator (ICD) devices (a.k.a., pacemaker devices) or artificial heart devices, for example.

As shown in FIG. 15, in one embodiment, the power module 140 includes a radio frequency antenna 142, a radio transceiver 144, a main carrier modem (modulation/demodulation device) 146, a FM modem 148, an analog-to-digital (A-D) converter 150, and a microcontroller 152 coupled to a power source 154. The antenna 142 and transceiver 144 enable communications with an external device via radio telemetry to control the power, duration and frequency of power supplied to one or more of the electromagnets 128 and/or 130. Incoming radio frequency (RF) command and/or data signals are demodulated by the modems 146 and 148 and supplied to the A-D converter, which converts analog waveforms into digital signals. The digital signals are then provided to microcontroller 152 for processing. The microcontroller 152 is coupled to the power source 154 for controlling the amount, duration, frequency, on/off times, etc. of the power source 154. The microcontroller 152 can further include a programmable read only memory (PROM) (not shown), a random access memory (RAM) and a microprocessor (not shown) for executing program instructions stored in the PROM, processing received data, storing any desired data, controlling the operation of the power source 154, and/or communicating with external devices (e.g. a monitor and/or controller with keypad). Each of the electronic components or modules 142-154 may be separate components or, alternatively, integrated into one or more integrated circuit (IC) chips.

In one embodiment, the power module 140 may be similar to the implantable power module (IPM) disklosed in U.S. Pat. No. 6,894,456, the entirety of which is incorporated by reference herein. Thus, the power module 140 can be configured to be implanted in a human for extended periods of time, and, contained within a hermetic biocompatible case having a highly reliable external connector (e.g., an external hermetic plug), a source of electrical power, a control circuit, an inductive recharging coil (in the case of secondary batteries and/or capacitors), a homing device for precisely locating the implanted module, and safety devices and design aspects to protect the patient in which the IPM is implanted. The source of power or “battery” may be one or more primary or secondary cells, a capacitor, a nuclear-heated thermopile or nuclear battery, or combinations of the above. In one embodiment, the source of power may include one or more lithium cells which provide good energy capacity, reliability, safety, and rate capability. However, hybrid devices having properties of both lithium cells and super capacitors may have improved performance depending on the demands of the device it is powering. While the term “battery” is used for convenience herein, its meaning may include any electrical storage or generation device.

It is further understood that the power module 140 need not continuously supply current to the electromagnets 128 and/or 130. Power may be supplied intermittently or only at desired times for desired durations while the power module 140 is sufficiently charged to provide a desired amount of current. In one embodiment, the microcontroller 152 may be programmed to monitor the amount of power available in battery 154 and communicate via RF telemetry to an external device when the battery 154 needs to be recharged (e.g., via magnetic induction). Such an external device (not shown) may be configured or integrated into a wrist-watch type of device, for example, so as to provide a convenient monitor to a patient without disrupting normal everyday activities. Other types of external devices (e.g., cell phones, personal digital assistants (PDA's), etc.) also may be advantageously utilized to communicate with and provide control signals to the power module 140. As battery technologies continue to improve, the power source 154 will be able to continuously provide power to the electromagnets 128 and/or 130 for longer periods of time before recharging is required. For example, the power source 154 may utilize thermoelectric nanomaterials that are adapted to store heat, e.g., from the patient's body, and convert that heat into electricity to be used for recharging the power source 140. Such thermoelectric nanomaterials are known in the art and discussed for example in, T. E. Humphrey and H. Linke, “Reversible Thermoelectric Nanomaterials,” Physical Review Letters, Engineering Physics, University of Wollongong, Australia (published Mar. 9, 2005), the entirety of which is incorporated by reference herein.

In a further embodiment, the microcontroller 152 may be programmed to monitor axial loads exerted onto the implant 120 via, for example, pressure sensors (not shown), and/or changes in magnetic flux between the electromagnets 128 and 130, or other permanent magnets (not shown), located on the first and second pieces 122 and 124. As axial loads increase, the first and second pieces 122 and 124 will be pushed closer together and the space between them will decrease. By measuring the change in distance between the first and second pieces 122 and 124, for example, the microcontroller 152 can monitor the axial loads exerted onto the implant 120. One exemplary method of measuring the spacing between the first and second pieces 122 and 124 is disklosed in U.S. publication no. 2005/0010301 A1, the entirety of which is incorporated by reference herein. By monitoring the axial loads exerted onto the device, the microcontroller 152 may turn on and/or control the level of current supplied to the electromagnets 128 and 130 and thereby provide a suitable distraction or repelling force between the electromagnets 128 and 130 to compensate for the increased axial loads. For example, when a patient is undergoing strenuous physical activity (e.g., running), the microcontroller 152 may sense increased axial loads on the spine and thereafter turn on the electromagnets 128 and 130 or increase the current level supplied to them in order to provide increased distraction or cushioning forces to compensate for the axial load changes. As another example, a patient may desire magnetic distraction forces to be applied to the patient's spine for only a few minutes or hours a day, in order to stretch and/or provide relief from compression and axial loads applied to the spine throughout the day.

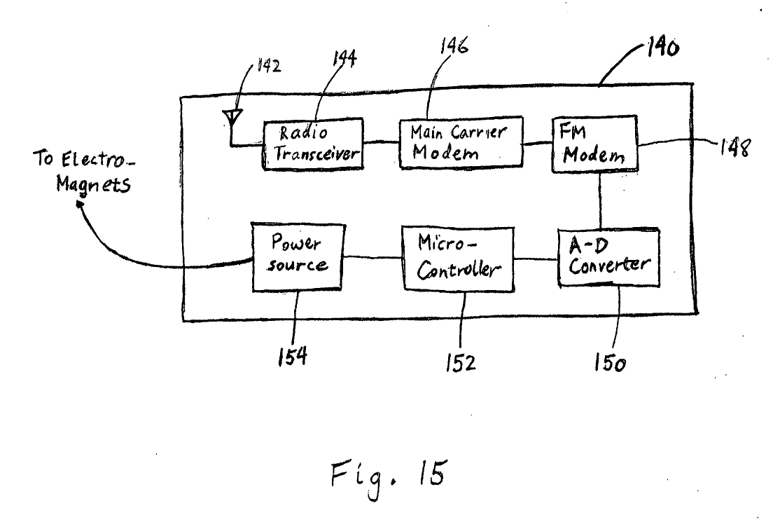

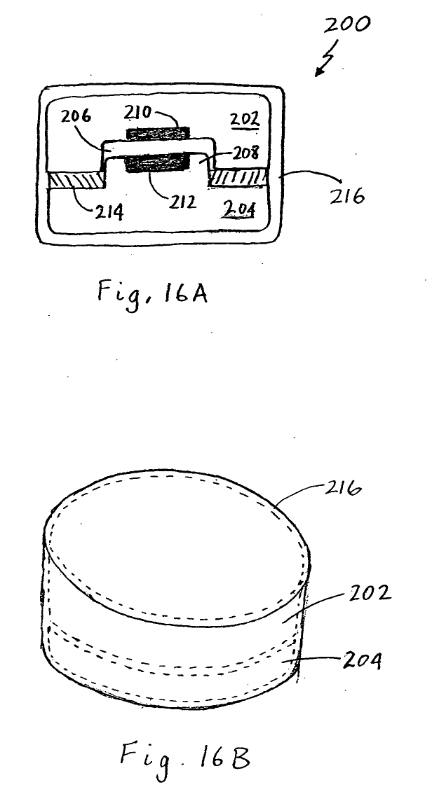

FIG. 16A illustrates a cross-sectional side view of a magnetic implant device 200, in accordance with another embodiment of the invention. The implant 200 includes a first piece 202 configured to be juxtaposed with a second piece 204. The first piece 202 includes a cavity or indentation 206 configured to receive a raised portion 208 of the second piece 204. The mating between the cavity 206 and the raised portion 208 facilitate alignment and resist undesired lateral movement of the first and second pieces 202 and 204. A first magnet 210 is embedded in the first piece 202 at or near a surface of the cavity 206 and adjacent a second magnet 212 embedded in the raised portion 208 of the second piece 204. In one embodiment, the first magnet 210 repels the second magnet 212 so as to provide a dynamic distraction force that increases as the first and second magnets 210 and 212 are brought closer together by external compression forces.

An annular gasket 214 surrounds the raised portion 208 of the second piece 204 and is similar to the gasket 36 described above with respect to FIGS. 2A and 2B above. In one embodiment, the gasket 214 is made from a compressible biocompatible materials (e.g., elastomer) that provides additional cushioning between the first and second pieces 202 and 204. The implant device 200 can further include an optional encasing or cover 216 that partially or completely encapsulates the implant device 200. One purpose of the encasing 216 is to hold the pieces 202 and 204 together and prevent undesired lateral shifting between the first and second pieces 202 and 204. In one embodiment, the encasing 216 also functions as an electromagnetic shield that prevents or reduces interference effects to and from external electromagnetic sources.

FIG. 16B illustrates a perspective external view of the implant 200, in accordance with one embodiment of the invention. In this embodiment, the implant 200 is shaped substantially in an oval configuration and designed to be implanted between two vertebral bodies or within a single vertebral body. The outer cover 216 entirely covers the first and second pieces 202 and 204 contained therein.

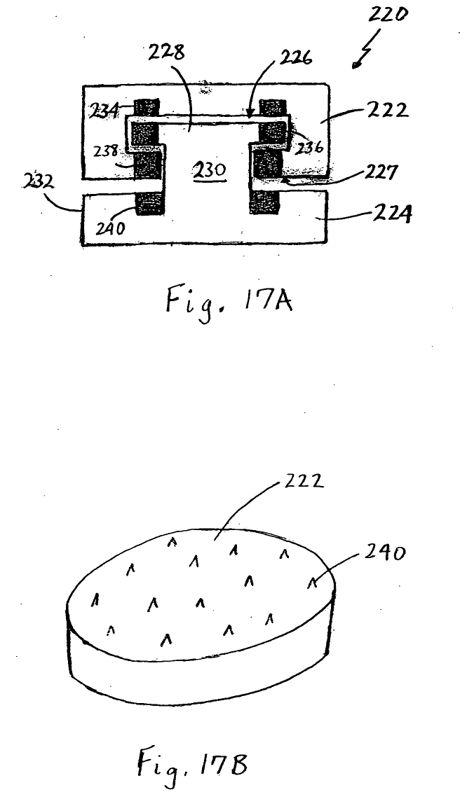

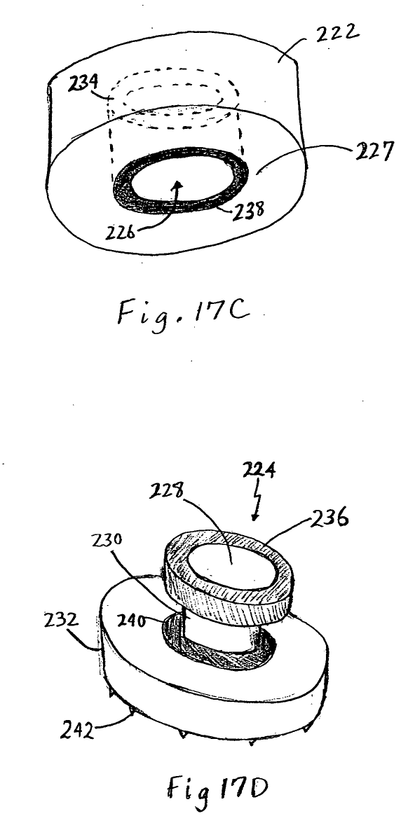

FIG. 17A illustrates a cross-sectional side view of a magnetic implant 220, in accordance with another embodiment of the invention. The implant 220 includes a first piece 222 that interlocks with a second piece 224 while allowing a limited range of motion between the first and second pieces 222 and 224. The first piece 222 includes an interior recess or chamber 226 that has a narrower opening formed by an internally flanged lip 227. An interlocking head portion 228 of the second piece 224 is positioned within the chamber 226 and prevented from exiting the chamber 226 by the internally flanged lip 227. The diameter or circumference of the interlocking portion 226 is larger than the opening formed by the flanged lip 227 such that it is captured within the chamber 226. However, the chamber 226 is sized to allow a desired amount of motion by the interlocking portion 226 within the chamber 226. In one embodiment, the chamber 226 and interlocking portion 228 are configured to only allow relative motion in the vertical direction. However, in other embodiments, they may be configured to allow limited motion in one or more of six degrees of freedom (e.g., x, y, z, roll, pitch and yaw). The interlocking portion 228 is coupled to one end of a column portion 230 which is sized to pass through the narrower opening formed by the internally flanged lip 227. A base portion 232 of the second piece 224 is coupled to the other end of the column portion 230.

A first annular magnet 234 is embedded or encased within the first piece 222 and juxtaposed with a second annular magnet 236 embedded or encased within the interlocking portion 228 of the second piece 224. In one embodiment, the first and second annular magnets 234 and 236 apply a repelling magnetic force on each other so as to distract the first and second pieces 222 and 224 away from each other. As discussed above, this distraction force dynamically increases as the magnets 234 and 236 are brought closer together. In one embodiment, the first and second magnets 234 and 236 are the only magnets present in the implant device 220. It will be understood that the first and second magnets need not be annular in shape but may be configured in the shape of disks or any other desired shape.

In a further embodiment, a third annular magnet 238 is optionally provided in the flanged lip portion 227 of the first piece 222. In one embodiment, this third annular magnet 238 is configured to provide a repelling force against the second annular magnet 236 so as to provide a counter-distraction force that resists the distraction force between the first and second magnets 234 and 236. Thus, a dynamic magnetic cushion is provided in both the distraction and counter-distraction directions of motion between the first and second pieces 222 and 224, respectively. In a further embodiment, a fourth annular magnet 240 may be embedded or encased within the base portion 232 of the second member to further provide a magnetic repulsion force to the third annular magnet 238, thereby providing an additional distraction force between the first and second pieces 222 and 224. It will be understood that the relative strengths and/or polarities of the magnets 234, 236, 238 and 240 may be selected and/or some magnets may be eliminated altogether to bias the implant device 220 in a desired manner such that it provides desired distraction or counter-distraction forces, for example, or behaves in accordance with a desired dynamic profile as the first and second pieces 222 and 224 move toward and away from each other.

FIG. 17B is a perspective view of the first piece 222 of FIG. 17A, in accordance with one embodiment of the invention. It is understood that other shapes and configurations of the first piece 222 are also possible. In one embodiment, a plurality of anchoring elements or spikes are provided on a top surface of the first piece 222 to provide improved grip or fixation when the first piece 222 is attached or positioned adjacent to a first spinal bone structure (not shown). FIG. 17C illustrates a perspective bottom view of the first piece 222, in accordance with one embodiment. The third annular magnet 238 is located at the internal peripheral edge of the flanged lip 227 and defines an opening to internal chamber 226, the shape of which is defined by the dashed lines, in accordance with one embodiment of the invention. The first annular magnet 234 which resides within the chamber 226 is also defined by dashed lines. In one embodiment, the flanged lip portion 227 is welded and/or screwed onto the rest of the first piece 222 after the interlocking portion 228 of the second piece 224 has been placed into the chamber 226.

FIG. 17D illustrates a perspective view of the second piece 224 of FIG. 17A, in accordance with one embodiment of the invention. In this embodiment, the interlocking portion 228 is cylindrical or disk shaped and coupled to a first end of a cylindrical column portion 230. The other end of the column portion 230 is coupled to the base portion 232, which also cylindrical or disk shaped. In one embodiment, each of these portions are formed separately and then assembled together. Various sizes of the portions 228, 230 and 232 may be mixed and matched to achieve desired size and functional configurations. For example, a column portion 230 of a desired length may be selected from a plurality of column portions of various lengths. Similarly, the interlocking portion 228 and base portion 232 may be selected from a variety of size and shape configurations. In one embodiment, the column portion 230 is threadingly engaged with both the interlocking portion 228 and the base portion 232. Thereafter, the portions may be welded or otherwise bonded together to form a substantially permanent second piece 222. In another embodiment, the column portion 230 is integrally formed with the interlocking portion 228. After the interlocking portion 228 is placed within the chamber 226 of the first piece 222, the flanged lip 227 is placed around the column portion 230 and welded onto the bottom of the first piece 222 to form the narrow opening of the chamber 226, thereby trapping the interlocking portion 228 within the chamber 222 but allowing the column portion 230 to slide back and forth through the opening. Thereafter, the base portion 232 is attached to the other end of the column portion 230 as described above, or by other known means.

The second annular magnet 236 is shown attached to the periphery of the interlocking portion 228. The magnet 236 may be attached or embedded within the interlocking portion 228 using any known means, such as welding, gluing, bonding, embedding within an annular groove or recess formed on the portion 228, or any combination of these techniques or other known techniques. Similarly, optional fourth annular magnet 240 is embedded within an annular recess or groove formed within the base portion 232 where the column portion 230 intersects the base portion 232. In one embodiment, the column portion 230 threadingly secures the fourth annular magnet 240 within the annular recess of the base portion 232. Thereafter, the column portion 230 may be welded to the base portion 232 on the opposite side of the base portion 232 to permanently fix these portions together. In a further embodiment, the opposite side of the base portion 232 includes a plurality of anchor elements or spikes 242 designed to resist movement or sliding of the second piece 224 with respect to an adjacent spinal structure.

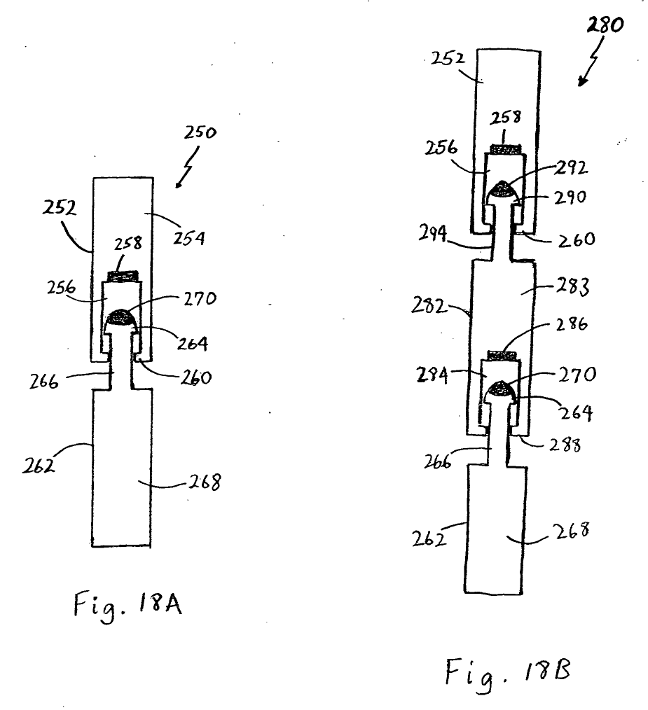

FIG. 18A is a cross-sectional side view of a magnetic spinal implant 250, in accordance with a further embodiment of the invention. The implant 250 includes a first piece 252 having a first end 254 configured to be received within and secured to a head of a pedicle screw (not shown), vertebral body screw (not shown) or other type of securing device. The first piece further includes a recess or chamber 256 at an end opposite to the first end 254. Embedded within or near an internal top surface of the chamber 256 is a first magnet 258. The first piece 252 also includes a flanged lip 260 at a bottom end of the first piece 252 that forms a relatively narrow opening to the chamber 256 such that the lateral diameter of the chamber 256 is greater than the diameter of the opening formed by the flanged lip 260.

The implant 250 also includes a second piece 262 configured to interlock with the first piece 252. The second piece 262 includes an interlocking portion 264 configured to be positioned within the chamber 256 and trapped therein by the flanged lip portion 260 of the first piece 252. A column portion 266 is coupled to the interlocking portion 264 at one end and coupled to a base portion 268 at its other end. In one embodiment, the column portion 266 is sized to snugly but smoothly pass through the flanged lip portion so as to facilitate alignment between the first and second pieces 252 and 262. Similarly, the interlocking portion 264 is sized to snugly but slidably fit within the chamber 256. The base portion 268 of the second piece 262 is configured to be received within and secured to a head of a second pedicle screw, vertebral body screw or other securing means. The second piece 262 includes a second magnet 270 located at or near the head of the interlocking member 264. In one embodiment, the first magnet 258 and second magnet 270 magnetically repel each other to provide a dynamic cushion against axial compression forces exerted on the spine.

As shown in FIG. 18A, the magnetic head portion 270 of the interlocking portion 264 is tapered so that it may be “snapped” into the chamber 256 and thereafter locked therein by the flanged lip portion 260. Thus, the first and second pieces 252 and 262 are configured to be engaged with each other in a “snap-lock” fashion. In one embodiment, the magnetic spinal implant 250 may be assembled to custom fit a particular patient's needs by providing a plurality of first pieces 252 of various sizes, configurations and/or magnetic strengths and a plurality of second pieces 262 of various sizes, configurations and/or magnetic strengths. Thus, a surgeon or other assembler of the device 250 can “mix and match” different first and second pieces 252 and 262 to achieve a desired overall size, configuration and magnetic strength. After first and second pieces 252 and 262 have been selected from the plurality of first and second pieces, the assembler can simply snap-fit the selected first and second pieces 252 and 262 together. This type of interlocking configuration is further intended to provide a limitation to excessive repulsion, attraction, or lateral slippage of the two portions, 252 and 262, thus controlling the range of motion of the magnetic implants.

FIG. 18B illustrates a multi-level magnetic implant 280, in accordance with a further embodiment of the invention. This implant 280 includes first and second pieces 252 and 262 described above with respect to FIG. 18A and further includes a center piece 282 that has elements of both the first and second pieces 252 and 262. The center piece 282 includes a main body portion 283 configured to be engage with a third pedicle screw, vertebral body screw or other bone securing device. The center piece 282 further includes a second interlocking portion 290 having a magnetic head portion 292, wherein the interlocking portion 290 is coupled to the main body portion 283 by a column portion 294. The head portion 292 is tapered so as to be “snap-fitted” within the chamber 256 of the first piece 252. In one embodiment, the magnets 292 and 258 will exert a magnetic repulsion force on each other.

At the opposite end of the center piece 282 is a chamber 284 similar or identical to chamber 256 of the first piece 252. An opening to the chamber 284 is defined by a flanged lip 288 that functions in an identical or similar manner as the flanged lip 260 of the first piece 252. Thus, the interlocking member 264 and magnetic head 270 is slidingly locked within the chamber 284, as described above with respect to FIG. 18A. Since the magnetic head 270 is tapered, it may be snap fit into the chamber 284, as described above. A magnet 286 is located near a top surface of the chamber 284 to provide a magnetic repelling force against the magnet 270 of the second piece 262. Each of the first, second and center pieces 252, 262 and 282, respectively, may be selected from a plurality of first, second and center pieces having various sizes, configurations and/or magnetic strengths, and thereafter snap-fitted together as discussed above. Thus, in one embodiment, the invention provides a multi-level implant 280 that can be custom tailored and fitted to meet the needs of a particular patient or application.

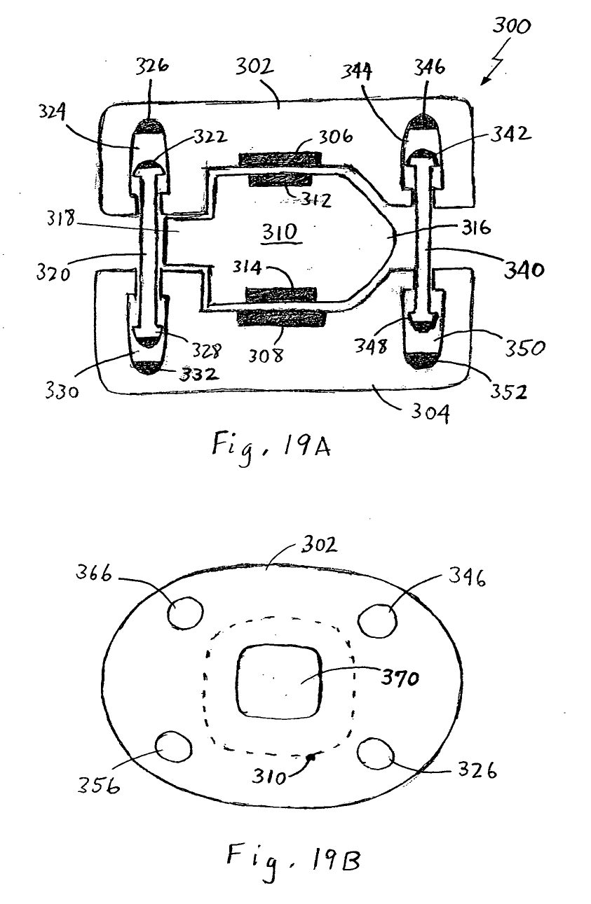

FIG. 19A is a cross-sectional side view of a three-piece expandable magnetic implant device 300, in accordance with another embodiment of the invention. The device 300 is similar to the implant 100 illustrated in FIGS. 12 and 13 and in various embodiments can be used for inter-vertebral disk replacement, vertebral body replacement, or intra-vertebral body augmentation, for example. As shown in FIG. 19A, the implant 300 includes a first piece 302 that includes a first magnet 306 and the second piece 304 includes a second magnet 308. An interchangeable center piece or spacer 310 includes two center magnets 312 and 314 wherein the center magnet 312 is configured to induce a magnetic force between itself and the first magnet 306 and the center magnet 314 is configured to induce a magnetic force between itself and the second magnet 308. In alternative embodiments, the center magnets 312 and 314 can be a single integral magnet instead of two separate magnets. The polarity of the magnets 306, 308, 312 and 314 can be selected such that the magnetic forces induced between magnets 312 and 306 and between magnets 314 and 308 can both be repelling forces, one repelling force and one attraction force, or both attraction forces. If both are repelling forces, this configuration provides a maximum level of distraction and shock absorption against axial compression forces. If one is attraction and the other is repulsion, this configuration provides an intermediate level of distraction while magnetically holding the spacer 310 against either the first or second piece 302 or 304, respectively. If both provide attraction forces, the interchangeable spacer 310 is magnetically attached and held to each of the first and second pieces 302 and 304 to provide maximum holding strength between the spacer 310 and each of the first and second pieces 302 and 304.

The implant 300 is different from the implant 100 described and illustrated in FIGS. 12 and 13 in that it includes a plurality of linking pins or rods, two of which are illustrated as 320 and 340 in FIG. 19A. The linking pins 320 and 340 are designed to prevent total separation between the first and second pieces 302 and 304 beyond a range determined by the length of the linking pins 320 and 340, and further prevent or limit relative lateral movement between the first and second pieces 302 and 304. In one embodiment, the first linking pin 320 includes a first magnetized and tapered head 322 configured to be snap-fit inserted into a first linking chamber 324 located in the first piece 302. A magnet 326 is located at a top surface of the linking chamber 324 such that a magnetic force is generated between the magnet 326 and the magnetic head 322. A second magnetized and tapered head 328 is located at an opposite end of the linking pin 320 and snap-fit locked within a second linking chamber 330 of the second piece 304. A magnet 332 in the second linking chamber 330 induces a magnetic field with the magnetic head 328. Similarly, the linking pin 340 includes similar tapered magnetic heads 342 and 348 at opposite ends thereof. The tapered magnetic heads 342 and 348 are configured to be inserted into respective third and fourth linking chambers 344 and 350 of the first and second pieces 302 and 304, respectively. Third and fourth magnets 346 and 352 within the respective chambers 344 and 350 generate respective magnetic forces between themselves and respective magnetic heads 342 and 348.

In one embodiment, the magnetic forces generated between the linking pin magnets 322, 328, 342 and 348 and their respective linking chamber magnets 326, 332, 346 and 352 are magnetic repulsion forces that can supplement or replace the repulsion forces generated between the spacer magnets 312 and 314 and respective first and second magnets 306 and 308. However, it will understood that various magnetic configurations may be implemented to achieve desired characteristics in terms of magnetic biasing and duration of the magnetic strength of some or all of the magnetic elements. For example, the spacer magnets may be selected to provide attraction forces while the linking pin magnets generate repulsion forces with their respective magnetic counterparts. Upon initial implantation, the attraction forces generated by the spacer magnets dominate such that the first and second pieces 302 and 304 are securely attached magnetically to the spacer 310. However, the spacer magnets may be selected (e.g., non-neodymium magnets) such that their magnetic properties deteriorate at a much faster rate than the linking pin magnets such that after a period of time the repulsion forces of the more permanent linking pin magnets (e.g., neodymium magnets) will dominate. In this way the magnetic biasing of the implant 300 may be dynamically changed over time without any further surgery or invasive procedure upon the patient. It will be appreciated that the magnetic biasing of the implant 220 of FIG. 17A can be similarly designed to change over time by appropriately selecting the type and strength of the magnets 234, 236, 238 and 240. Various magnetic biasing configurations will be apparent to those of skill in the art.

FIG. 19B illustrates a cross-sectional top view of the first piece 302 of the implant 300, in accordance with one embodiment. In this embodiment, the first piece 302 is configured in the shape of an oval having four linking chamber magnets 326, 346, 356 and 366 and corresponding linking chambers (not shown) located near opposing peripheral edges of the first piece 302. An exemplary interchangeable spacer 310, indicated by dashed lines, may be inserted between the first and second pieces 302 and 304 by inserting through the space between chamber magnets 326 and 356, for example. Exemplary tools, methods and techniques for inserting the spacer 310 between the first and second pieces 302 and 304 are described in detail in the '003 application, the entirety of which is incorporated by reference herein. In one embodiment, the implant 300 includes at least one window or passage 370 in which bone growth material may be inserted to promote fusion between two vertebra located on opposite sides of the implant 300. The window 370 is shown in the first piece 302 in FIG. 19B. A similarly shaped and sized window would be present at corresponding locations of the second piece 304 and the spacer 310 to provide a passage from a top surface of the first piece 302 to a bottom surface of the second piece 304.

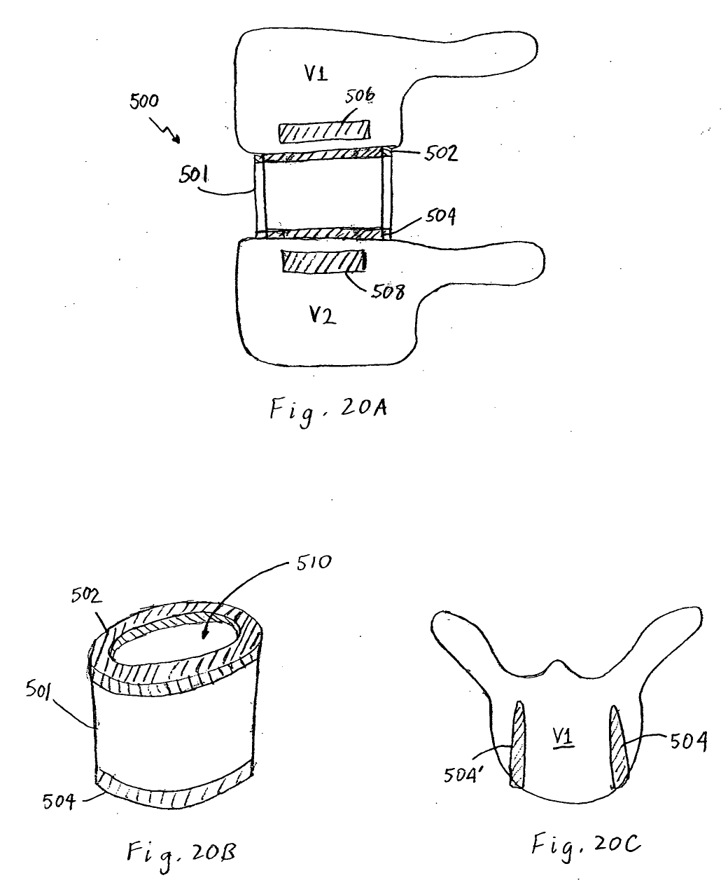

FIG. 20A illustrates a cross-sectional side view of a magnetic fusion implant system 500 positioned between two vertebra V1 and V2 to be fused, in accordance with a further embodiment of the invention. The system 500 includes a cylindrical or near-cylindrical housing 501 having a first annular magnet 502 located at a top opening of the cylindrical housing 501 and second annular magnet 504 located at a bottom opening of the cylindrical housing 501. The first annular magnet 502 is configured to be magnetically attracted to one or more first magnetic implants 506 embedded into the first vertebral body V1 at or near the bottom surface of the vertebra. The second annular magnet 504 is configured to be magnetically attracted to one or more second magnetic implants 508 embedded into the second vertebral body V2 at or near the top surface of the vertebra. The housing 501 is hollow or cylindrical so as to allow bone graft material to be packed therein to allow fusion between the first and second vertebras V1 and V2. FIG. 20B illustrates a perspective view of the housing 501 including top and bottom annular magnets 502 and 504, respectively, and an internal chamber or cavity 510 for receiving bone graft material therein. The implants 506 and 508 may be embedded or attached to their respective vertebras in any one of various known ways. In such various embodiments, the implants 506 and 508 may be magnetic or magnetized staples, pins, rods or screws (e.g., pedicle screws). FIG. 20C shows a cross-sectional top view of the first vertebra V1 having two magnetized pins 504 and 504′ implanted therein.

After the cavity 510 of the housing 501 is packed with bone graft material (not shown), it is placed between first and second vertebras V1 and V2. The magnetic attraction forces pull the first and second vertebras V1 and V2 against top and bottom magnets 502 and 504, respectively. This compresses the bone graft material between the vertebras and thereby causes load sharing, which increases the fusion rate. Additionally, the magnetic attraction forces limit or resist over-separation of the vertebras V1 and V2, which may cause the bone graft to become loose or dislodged from its position between the vertebras. In a further embodiment, the magnets 502, 504, 506 and 508 are selected and designed to be impermanent such that their magnetic field strengths decrease over a desired period of time, or such that they may be de-magnetized, after which the natural fusion of the adjacent vertebras will hold the vertebras together. This allows a more natural fusion mass to form between the vertebras that will adjust during the growth process to better match the adjacent bone characteristics. Thus, the magnetic implant system 500 functions to primarily hold the vertebras V1 and V2 together and compress the fusion mass at the beginning of the fusion process but as the magnetic strengths decrease, the natural fusion mass takes on greater responsibility for holding the vertebrae together. In various embodiments, the housing may be made from any suitable biocompatible materials or combination of materials (e.g., metals, PEEK, polymers, elastomers, etc.). In one embodiment, the housing 501 is made from a resilient elastomer designed to elastically pull the vertebras V1 and V2 toward each other. In this embodiment, the annular magnets 502 and 504 are encapsulated inside housing material near the top and bottom edges of the cylindrical elastomer housing 501.

While various embodiments of the present invention have been described above, it should be understood that they have been presented by way of example only, and not of limitation. Likewise, the various figures depict exemplary embodiments and configurations of the invention, which is done to aid in understanding the features and functionality that can be included in the invention. The invention is not restricted to the illustrated example configurations, but can be implemented using a variety of alternative architectures and configurations. Additionally, although the invention is described above in terms of various exemplary embodiments and implementations, it should be understood that the various features and functionality described in one or more of the individual embodiments are not limited in their applicability to the particular embodiment with which they are described, but instead can be applied, alone or in some combination, to one or more of the other embodiments of the invention, whether or not such embodiments are described and whether or not such features are presented as being a part of a described embodiment. Thus the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as mean “including, without limitation” or the like; the term “example” is used to provide exemplary instances of the item in discussion, not an exhaustive or limiting list thereof; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the invention may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

What is claimed is:

1. A magnetic device, comprising:
a first piece configured to be implanted into a patient and coupled to the patient's spine, wherein the first piece includes a recessed portion;
a first magnet coupled to the first piece;
a second piece configured to be implanted into the patient and juxtaposed with the first piece, wherein the second piece includes a base portion surrounding a raised portion, wherein the raised portion is configured to be at least partially received within the recessed portion of the first piece so as to facilitate alignment of the first and second pieces with respect to one another; and
a second magnet coupled to the second piece, wherein the first magnet exerts a desired magnetic force on the second magnet.

2. The device of claim 1 wherein the desired magnetic force is a repelling force.

3. The device of claim 1 wherein the desired magnetic force is an attracting force.

4. The device of claim 1 further comprising a compressible member located on the base portion and surrounding the raised portion of the second piece.

5. The device of claim 1 further comprising a cover configured to hold the first and second pieces together and resist undesired lateral movement of the first and second pieces with respect to one another.

6. The device of claim 5 wherein the cover is made from an electromagnetic shielding material.

7. The device of claim 1 wherein at least one of the first and second magnets comprises an electromagnet.

8. The device of claim 7 further comprising an implantable power source coupled to the at least electromagnet.

9. The device of claim 1 wherein the indented portion in the first piece comprises a chamber having an opening defined by a flanged lip portion of the first piece and the raised portion of the second piece comprises an interlocking member configured to be retained within the chamber by the flanged lip portion such that the first and second pieces are interlocked with each other with a limited range of motion being allowed between the first and second pieces.

10. The device of claim 9 wherein the second magnet is located on the interlocking member.

11. The device of claim 10 further comprising a third magnet located on the flanged lip portion of the first piece so as to exert a counter-acting magnetic force on the second magnet that counteracts the magnetic force generated between the first and second magnets.

12. The device of claim 10 wherein the first magnet is located on the flanged lip portion of the first piece and the device further comprises a third magnet located on the base portion of the second piece so as to exert a counter-acting magnetic force on the first magnet that counteracts the magnetic force generated between the first and second magnets.

13. A magnetic device, comprising:
a first piece configured to be implanted into a patient and coupled to the patient's spine;
a first magnet coupled to the first piece;
a second piece configured to be implanted into the patient and juxtaposed with the first piece;
a second magnet coupled to the second piece so as to exert a desired magnetic force on the first magnet, wherein at least one of the first and second magnets comprises an electromagnet; and
a power source coupled to the at least one electromagnet.

14. The device of claim 13 wherein the power source is configured to be fully implanted into the patient.

15. The device of claim 13 further comprising a microcontroller coupled to the power source for controlling an electrical current provided by the power source to the at least one electromagnet.

16. The device of claim 15 further comprising a radio frequency transceiver device coupled to the microcontroller so as to allow the microcontroller to receive radio frequency command signals from a device external to the patient's body.

17. A magnetic device, comprising:
a first piece configured to be implanted in a patient and coupled to the patient's spine;
a first magnet coupled to the first piece;
a second piece configured to be implanted in the patient and coupled to the patient's spine;
a second magnet coupled to the second piece;
a spacer configured to be placed between the first and second pieces; and
at least one spacer magnet coupled to the spacer so as to exert magnetic forces on the first and second magnets.

18. The device of claim 17 further comprising means for interlocking the first and second pieces so as to allow a limited range of motion between the first and second pieces.

19. The device of claim 17 wherein at least one of the first, second, and at least one spacer magnets comprises an electromagnet, the device further comprising:
a power source is configured to be fully implanted into the patient and coupled to the at least one electromagnet; and
a microcontroller coupled to the power source for controlling an electrical current provided by the power source to the at least one electromagnet.

20. The device of claim 19 further comprising a radio frequency transceiver device coupled to the microcontroller so as to allow the microcontroller to receive radio frequency command signals from a device external to the patient's body.
